(19) United States
(12) Patent Application Publication
(10) Pub. No.: US 2023/0241090 A1
(43) Pub. Date: Aug. 3, 2023

(54) CANCER CELL PROLIFERATION INHIBITOR AND HEALTH FOOD

(71) Applicant: FORDAYS CO., LTD., Tokyo (JP)

(72) Inventors: Akiko YUASA, Osaka-shi, Osaka (JP); Isao YUASA, Osaka-shi, Osaka (JP); Keita SUTOH, Tokyo (JP); Keisuke KIRIYAMA, Tokyo (JP)

(21) Appl. No.: 18/004,895

(22) PCT Filed: Jul. 9, 2021
(86) PCT No.: PCT/JP2021/026025

§ 371 Date: Jan. 10, 2023

(30) Foreign Application Priority Data
Jul. 17, 2020 (JP) 2020-123184

(51) Int. Cl. A61K 31/7105; A61K 36/064; A61P 35/00; A23L 33/00; A23L 33/13; A23L 33/145

(52) U.S. Cl. CPC A61K 31/7105; A61K 36/064; A61P 35/00; A23L 33/40; A23L 33/13; A23L 33/145

(57) ABSTRACT

The present invention provides a cancer cell proliferation inhibitor and a health food, suppressing cancer cell proliferation by using nucleic acid and RNA. A cancer cell proliferation inhibitor and a health food containing RNA extracted from torula yeast can inhibit a cell cycle of a cancer cell.

### TECHNICAL FIELD

The present invention relates to a cancer cell proliferation inhibitor and a health food, and more specifically to a cancer cell proliferation inhibitor and a health food, containing yeast-derived RNA. Note here that the health food is intended to include a drink.

### BACKGROUND ART

In recent years, reflecting an increase in public interest in health, health foods using nucleic acid, deoxyribonucleic acid (DNA), ribonucleic acid (RNA), or nucleoprotein as a raw material or an active component have been provided.

For example, nucleic acids, particularly RNA, extracted from yeast are known as nutrients that aid cell metabolism. Furthermore, there is a description that the nucleic acid and DNA of salmon milt have an advantageous effect of suppressing cancer cell proliferation, and the mechanism of suppressing cancer cells acts in the G2/M phase of the cell cycle (Patent Document 1).

### Citation List

### Patent Document

Patent Document 1: Japanese Patent No. 6153736

### SUMMARY OF THE INVENTION

### Technical Problems

The present inventors have engaged in elucidating a mechanism of suppressing cancer cell proliferation by RNA. Then, based on the findings, the present invention provides a cancer cell proliferation inhibitor and a health food, acting on the mechanism elucidated so as to suppress cancer cell proliferation.

### Solution to Problem

In order to solve the above-mentioned problems, the present inventors have intensively studied the advantageous effect of RNA on tumor cells. As a result, the present inventors have found that the progression degree of the ascites carcinoma of a tumor-bearing mouse produced by inoculation of Ehrlich ascites tumor cell EATC depends on intake of RNA and that the progression of the carcinoma is suppressed in the group taking RNA. Furthermore, the present inventors have found that the progression from the G1 phase to the S phase in the cell cycle of a cancer cell is inhibited as a suppressing action mechanism of the progression of the carcinoma, and have completed the present invention.

In other words, one embodiment of the present invention relates to:
1. a cancer cell proliferation inhibitor that is a formulation for inhibiting progression of a cell cycle of a cancer cell, containing a yeast-derived RNA as an active component;
2. the cancer cell proliferation inhibitor described in 1, wherein the yeast-derived RNA is an RNA extracted from torula yeast;
3. the cancer cell proliferation inhibitor described in 1 or 2, wherein the yeast-derived RNA is an RNA that inhibits progression from a G1 phase to an S phase in the cell cycle of the cancer cell;
4. a health food for inhibiting progression of a cell cycle of a cancer cell, containing a yeast-derived RNA as an active component;
5. the health food described in 4, wherein the yeast-derived RNA contains an RNA extracted from torula yeast; and
6. the health food described in 4 or 5, wherein the yeast-derived RNA includes an RNA that inhibits progression from a G1 phase to an S phase in the cell cycle of the cancer cell.

Furthermore, another embodiment of the present invention can include a method for suppressing progression of cancer by orally administering the cancer cell proliferation inhibitor and the health food to a subject, and use of the cancer cell proliferation inhibitor and the health food for suppressing progression of cancer.

### Advantageous Effect of Invention

The present invention provides a cell proliferation inhibitor and a health food, having very high safety and few side effects.

Furthermore, the cell proliferation inhibitor and the health food of the present invention function in a G1 phase of a cell cycle and inhibits progression of the cell cycle to an S phase, so that proliferation potency of a cancer cell can be suppressed in an early stage of the cell cycle.

### BRIEF DESCRIPTION OF THE DRAWINGS

FIG. 1 is a view showing a cell cycle in a cell division, showing a relationship among G1, S, G2, and M phases.

FIG. 2 is a schematic view showing how proteins and the like are related in a G1/S checkpoint (a transition point from the G1 phase to the S phase).

FIG. 3 is a graph showing an effect of a control and RNAs (400 μg/mL and 800 μg/mL) on the number of living cells of mouse fibroblasts (3T3-L1, normal cells).

FIG. 4 is a graph showing an effect of RNAs (50, 100, 200, and 400 μg/mL) on the number of living cells of hepatocytes (normal cells).

FIG. 5 is a graph showing an effect of a control and RNAs (50, 100, 200, and 400 μg/mL) on the cell viability (%) of mouse-derived Ehrlich ascites tumor cells (EATC).

FIG. 6 is a graph showing an effect of a control and RNAs (50, 100, 200, and 400 μg/mL) on the cell proliferation potency of mouse-derived Ehrlich ascites tumor cells (EATC) by measuring the number of cells (×10⁶ cells/mL).

FIG. 7 is a graph showing an effect of a control and RNAs (200, and 400 μg/mL) on the DNA synthesis ability by calculating the BrdU positive cells (%).

FIG. 8 is a graph showing an effect of a control and RNA (400 μg/mL) on the expression of highly phosphorylated Rb protein (ppRb) in terms of the relative strength of pRb and ppRb.

FIG. 9 is a graph showing an effect of a control and RNA (400 μg/mL) on the expression of cyclin E protein in terms of the relative strength of cyclin E to β-actin.

FIG. 10 is a graph showing an effect of a control and RNA (400 μg/mL) on the expression of p21 in terms of the relative strength of p21 to β-actin.

FIG. 11 is a graph showing an effect of a control and RNA (400 μg/mL) on the expression of p53 in terms of the relative strength of p53 to β-actin.

FIG. 12 is a graph showing an effect of a control and RNA (mouse to which RNA was administered) on an amount (mL) of the mouse ascites.

### DESCRIPTION OF EMBODIMENTS

The present invention will be described in more details.

Nucleic acid is a general term for DNA (deoxyribonucleic acid) that holds genetic information and RNA (ribonucleic acid) that transmits genetic information and synthesizes proteins according to the information held by DNA. Nucleic acid plays an important role in cell proliferation and growth. The food component described in the present invention contains a high content of nucleic acid of torula yeast. Among foods, torula yeast contains a particularly large amount of nucleic acid. Torula yeast is recognized as safe by the U.S. Food and Drug Administration (FDA), and its cell body is produced using sugar such as waste pulp liquid and molasses. Nucleic acid (RNA) extracted from the cell body is used for health foods. In the present invention, the effect of torula yeast extract on cancer cells was studied.

Cancer is a disease in which genes of normal cells continue to be damaged and become abnormal due to carcinogens, reactive oxygen species, viruses, and the like, resulting in indefinite cell proliferation and adversely affecting organs throughout the body. Although there is no absolute prevention method for cancer, it is thought that there is a strong correlation between diet and the development of cancer, and that cancer can be prevented by improving dietary intake. It is therefore important to search for food components having anticancer action.

There are two types of anticancer action: cell proliferation suppressing action of stopping cell cycle progression, and action of inducing apoptosis cell death.

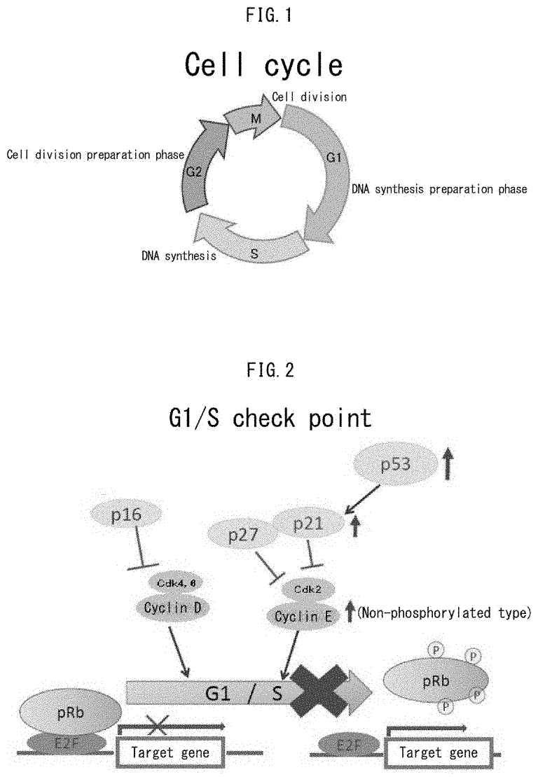

As shown in FIG. 1, cells proliferate by repeating an S phase in which cells replicate DNA; an M phase in which cells divide; and phases for preparing the above-mentioned phases (a G1 phase before the S phase and a G2 phase before the M phase). Cells that stop dividing are out of these phases. Such cells enter the cell cycle from the G1 phase when restarting dividing. The progression of the cell cycle is controlled by a complex of cyclins and Cdks (cyclin dependent kinase), which function as a promoting factor, and their regulatory proteins (Cdk inhibitory proteins, CDC25 family, WEE1 family, and the like). Cyclin/Cdk complexes allow the cell cycle to progress by phosphorylating target proteins at respective stages. In the cell cycle, the G1 phase is an important phase which includes an R point (a restriction point) and determines whether the cell will proliferate or undergo differentiation, senescence, or apoptosis. Cell proliferation needs a proliferation factor, but once cells pass the R point, they divide and proliferate regardless of the presence or absence of the proliferation factor. The Rb (retinoblastoma) gene is a cancer suppressor gene that encodes the Rb protein (pRb) having an effect of suppressing cell proliferation in the G1 phase. Mutations of the Rb gene have been identified in many cancers including retinoblastoma and osteosarcoma. Phosphorylation of pRb is thought to define the R point. pRb is a protein having a molecular weight of 110 kDa to 116 kDa. The pRb forms a complex with an E2F transcription factor and binds to the promoter of the gene, thereby suppressing gene transcription required for transition from the G1 phase to the S phase. Phosphorylation of pRb releases E2F from the complex, and E2F promotes transcription required for E2F the progression to the S phase. Phosphorylation of pRb is promoted by the CyclinD/Cdk4,6 complex and CyclinE/Cdk2 complex. Furthermore, in upstream of the complexes, a Cdk inhibitor (CKI (cyclin dependent kinase inhibitor)), which inhibits action of Cdk4, 6, or Cdk2, is present, and binds to the complexes so as to lose the phosphorylation ability of Cdk with respect to the Rb protein, resulting in suppressing the progression of the cell cycle. CKI is divided into two families: the INK4 family and the CIP/KIP family. The INK4 family includes p15, p16, p18, and p19. The CIP/KIP family includes p21, p27, and p57. The p21 is transcriptionally induced mainly by the cancer suppressor gene product p53 when DNA is damaged.

FIG. 2 is a schematic view showing the relationship of proteins at the G1/S checkpoint, and shows the relationship of proteins in shifting from the G1 phase to the S phase. Progression to the S phase needs high phosphorylation of Rb (ppRb). Many pRbs are required to be expressed for suppressing the cell division and inhibiting the progression to the S phase. For determination thereof, the pRb/ppRb ratio is analyzed.

Furthermore, it is shown that the increase in Cdk2 and cyclin E which negatively control the activation from the upstream as well as p21, p27, and p57 relating to Cdk2 and the like and p53 relating to p21 (negative control by the increase) prevents phosphorylation of pRb, and suppresses the progression into the S phase.

Therefore, examples analyze the pRb/ppRb ratio, and the relative strength of protein such as p21, thereby elucidating a mechanism in which the progression from the G1 phase to the S phase in the cell cycle is suppressed by administration of nucleic acid.

In one embodiment of the present invention, the cell proliferation inhibitor and the health food use RNA of torula yeast, but RNA is not limited to this. RNA from yeast other than torula yeast can be used. Specific examples of the other yeast include RNA extracted from beer yeast, torula yeast, milk yeast, and baker's yeast.

The active component of the cancer cell proliferation inhibitor of the present invention is torula yeast-derived RNA. Hereinafter, the description “cancer cell proliferation suppressing substance” represents yeast-derived RNA.

Examples of the administration form of the cancer cell proliferation inhibitor of the present invention include parenteral administration by injection (subcutaneous, intravenous, intramuscular, or intraperitoneal injection), ointment, suppository, aerosol, and the like, or oral administration by tablets, capsules, granules, pills, syrups, solutions, emulsions, suspensions, and the like.

The cancer cell proliferation inhibitor of the present invention contains the cancer cell proliferation suppressing substance as the active component at about 0.01 to 99.5% by mass, and preferably about 0.1 to 30% by mass with respect to the mass of all compositions.

The cancer cell proliferation inhibitor of the present invention can include other pharmaceutically or veterinary active compounds in addition to the cancer cell proliferation suppressing substance as an active component.

The clinical dose of the cancer cell proliferation suppressing substance included in the cancer cell proliferation inhibitor of the present invention varies depending on age, body weight, sensitivity of a patient, degree of symptoms, and the like, but the ordinarily effective dose is about 0.001 to 5.0 g, and preferably about 0.5 to 2.5 g per day for adults. However, dose out of the above range can be used, if necessary.

The cancer cell proliferation inhibitor of the present invention is formulated for administration by conventional pharmaceutical means.

In other words, tablets, capsules, granules, and, pills for oral administration are prepared using excipients such as sucrose, lactose, glucose, starch, mannitol; binders such as hydroxypropylcellulose, syrup, gum arabic, gelatin, sorbitol, tragacanth, methylcellulose, polyvinylpyrrolidone; disintegrants such as starch, carboxymethylcellulose or calcium salts thereof, microcrystalline cellulose, polyethylene glycol; lubricating agents such as talc, magnesium stearate or calcium, silica; lubricants such as sodium laurate, and glycerol, and the like.

Injections, solutions, emulsions, suspensions, syrups, and aerosols are prepared using solvents for the active components, such as water, ethyl alcohol, isopropyl alcohol, propylene glycol, 1,3-butylene glycol, polyethylene glycol; surfactants such as sorbitan fatty acid esters, polyoxyethylene sorbitan fatty acid esters, polyoxyethylene fatty acid esters, polyoxyethylene ethers of hydrogenated castor oil, lecithin; suspensions such as carboxymethyl sodium salts, cellulose derivatives such as methyl cellulose, and natural gums such as tragacanth and gum arabic; preservatives such as an ester of parahydroxybenzoic acid, benzalkonium chloride, and sorbate, and the like.

As the ointment of the transdermal absorption preparation, for example, white petrolatum, liquid paraffin, higher alcohol, macrogol ointment, hydrophilic ointment, aqueous gel base, and the like, are used.

The suppository is prepared by using, for example, cocoa butter, polyethylene glycol, lanolin, fatty acid triglyceride, coconut oil, polysorbate, or the like.

Formulation Examples of the cancer cell proliferation inhibitor of the present invention are shown below.

### Formulation Example 1

Tablet

| | | |
|---|---|---|
| Cancer cell proliferation suppressing substance | 20 | g |
| Lactose | 260 | g |
| Microcrystalline cellulose | 600 | g |
| Corn starch | 350 | g |
| Hydroxypropylcellulose | 100 | g |
| CMC-Ca | 140 | g |
| Magnesium stearate | 30 | g |
| Total amount | 1,500 | g |

The above-mentioned components were mixed by a routine procedure to produce 10,000 sugar-coated tablets each containing 1 mg of active component.

### Formulation Example 2

Capsule

| | | |
|---|---|---|
| Cancer cell proliferation suppressing substance | 20 | g |
| Lactose | 430 | g |
| Microcrystalline cellulose | 1,000 | g |
| Magnesium stearate | 50 | g |
| Total amount | 1,500 | g |

The above-mentioned components were mixed by a routine procedure, and the mixture was filled in gelatin capsules to produce 10,000 capsules each containing 1 mg of active component.

### Formulation Example 3

Soft capsule

| | | |
|---|---|---|
| Cancer cell proliferation suppressing substance | 25 | g |
| PEG400 | 464 | g |
| Saturated fatty acid triglyceride | 1,500 | g |
| Peppermint oil | 1 | g |
| Polysorbate 80 | 10 | g |
| Total amount | 2,000 | g |

The above-mentioned components were mixed, and the mixture was filled in No. 3 soft gelatin capsules by a routine procedure to produce 10,000 capsules each containing 1 mg of active component.

### Formulation Example 4

Ointment

| | | |
|---|---|---|
| Cancer cell proliferation suppressing substance | 1.0 | g |
| Liquid paraffin | 10.0 | g |
| Cetanol | 20.0 | g |
| White petrolatum | 68.4 | g |
| Ethylparaben | 0.1 | g |
| 1-menthol | 0.5 | g |
| Total amount | 100.0 | g |

The above-mentioned components were mixed by a routine procedure to obtain 1% ointment.

### Formulation Example 5

Suppository

| | | |
|---|---|---|
| Cancer cell proliferation suppressing substance | 1 | g |
| Witepsol H15* | 478 | g |
| Witepsol W35* | 520 | g |
| Polysorbate 80 | 1 | g |
| Total amount | 1,000 | g |

*Witepsol is a trade name of a triglyceride compound.

The above-mentioned components were melted and mixed by a routine procedure, and the resulting mixture was poured in a suppository container and solidified by cooling to prepare 1,000 suppositories each containing 1 mg of active component.

### Formulation Example 6

Injection

| | |
|---|---|
| Cancer cell proliferation suppressing substance | 1 mg |
| Distilled water for injection | 5 mL |

The injection was used in form of a solution at the time of use.

The present invention further relates to a health food including a cancer cell proliferation suppressing substance.

The active component in the health food of the present invention is a cancer cell proliferation suppressing substance, that is, torula yeast-derived RNA.

The health food of the present invention is suitably carried out as the health food having a cancer cell proliferation suppressing action. Furthermore, the health food may be products suited to tastes of users by mixing with various known components such as a sweetener, an acidulant, a vitamin, and the like. For example, the health food can be provided in the form of a tablet, a capsule, a drink, a dairy product such as yogurt, a seasoning, a processed food, a dessert, a confectionery, or the like.

The production process of these health foods is not particularly limited, and, for example, objective health foods can be produced by adding the above-mentioned sweetener and the like by appropriate means during processing of health foods. The cancer cell proliferation suppressing substance can be incorporated in a range of 1 mg to 20 g with respect to 100 g of the food.

Examples of the specific substances that can be added to the health food of the present invention include the followings, but not limited thereto. As collagen, porcine collagen peptide, fish collagen peptide (including gelatin), and collagen-containing mineral complex, and the like. The collagens can be used alone as a mixture of but as a mixture of two types or more of collagens.

Examples of chondroitin include chondroitins including chondroitin, which is a kind of glycosaminoglycan (mucopolysaccharide) and has a basic structure in which sulfuric acid is bonded to a sugar chain including repeating disaccharides of D-glucuronic acid (GlcA) and N-acetyl-D-galactosamine (GalNAc), the derivative thereof, and the salts thereof.

Examples of hyaluronic acid include hyaluronic acids including hyaluronic acid, which is a kind of proteoglycan and has a basic structure of linking disaccharide units in which the first position of β-D-glucuronic acid and the third position of β-D-N-acetyl-glucosamine are bonded, derivatives thereof and salts thereof and low molecular-weight hyaluronic acid or hyaluronic acid decomposition products obtained by subjecting such hyaluronic acids to enzymatic treatment using, for example, hyaluronidase, or to heating and pressurizing treatment. Specific examples of hyaluronic acid that can be added to the health food include a cockscomb extract, and the like.

Zinc is not particularly limited as long as it can be added in a form usable for foods, and can be administered in a form such as zinc gluconate, zinc sulfate, and edible zinc yeast.

Vitamins are not particularly limited as long as they are vitamins, derivatives thereof, or salts thereof, which can exhibit the advantageous effects of the present invention. Examples of vitamins include vitamin C (ascorbic acid), vitamin B1 (thiamine), vitamin B2 (riboflavin), vitamin B6 (pyridoxine), vitamin B12 (cobalamin), folic acid (vitamin B9), niacin (vitamin B3), calcium pantothenate, and the like.

The other components include sweeteners such as fructose glucose liquid sugar, syrup containing rare sugar, erythritol, and sucralose, fruit juices such as pineapple juice, preservatives such as sodium benzoate, colorants such as caramel dye, emulsifiers (for example, derived from soybean), perfumes, acidifiers, and the like. When the health food of the present invention contains these other components, an appropriate amount of each component can be added.

Examples of the components of the health food of the present invention are shown below.

Components included in health food (drink) (amount per 720 mL)

| Cancer cell proliferation suppressing substance | 1000 mg to 7000 mg | |
|---|---|---|
| Collagen | 75 | g |
| Chondroitin | 164 | mg |
| Hyaluronic acid | 64.8 | mg |
| Zinc | 720 | mg |
| Folic acid | 2.88 | mg |
| Niacin | 144 | mg |
| Vitamin C | 3600 | mg |
| Vitamin B1 | 13.68 | mg |
| Vitamin B2 | 14.4 | mg |
| Vitamin B6 | 15.84 | mg |
| Vitamin B12 | 25.92 | mg |
| Calcium pantothenate | 79.2 | mg |

| | |
|---|---|
| Other additive components | q.s. |

(Other additive components: fructose glucose liquid sugar/syrup containing rare sugar/erythritol/fruit juices such as pineapple juice, and the like)

In Test Example 5, in order to examine the advantageous effect in a living body, in vivo system was studied. A tumor-bearing mouse model was prepared by intraperitoneal administration of mouse-derived Ehrlich ascites tumor cells, and the effect of oral administration of RNA was studied. Cancers associated with ascites include liver cancer, pancreatic cancer, bile duct cancer, colorectal cancer, gastric cancer, peritoneal dissemination, carcinomatous peritonitis, ascites carcinoma, ovarian cancer, uterine cancer, and breast cancer. Diseases causing ascites include hepatitis C, cirrhosis, alcoholic hepatitis, general liver disease, inflammation of pancreas, bacterial peritonitis, tuberculous peritonitis, kidney disease, renal failure, heart failure, and malnutrition.

### EXAMPLES

Hereinafter, one embodiment of the present invention is specifically described with reference to examples, but the present invention is not limited to the following examples. Note here that samples, culture media, and cells, and the like, which were used in the following examples are as follows.

### (1) Sample

Torula yeast extract (RNA) was obtained from Fordays Co., Ltd.

### (2) Culture Medium

### 2-1. Preparation of Culture Medium

Dulbecco's Modified Eagle Medium (hereinafter, which will be referred to as DMEM medium, Nissui Pharmaceutical Co., Ltd.) was sterilized by autoclaving, and 0.1% penicillin, 0.1% streptomycin, and filter-sterilized 2% L-glutamine (584 mg/L) were added thereto. Then, pH thereof was adjusted to pH 7.5 using sterile baking soda.

### 2-2. Preparation Method of Sterile Sodium Bicarbonate

Into each of heat-treated (at 200° C. for 30 minutes) ampoule tubes, 5 mL of 8% NaHCO₃ solution (40 g/500 mL H₂O) was dispensed. The ampoule tubes were sealed using a burner, and then subjected to autoclaving.

### 2-3. Inactivation of FBS

Fetal bovine serum (FBS) was purchased from Sigma Corporation. The serum includes a complement. Activation of the complement causes cytotoxicity. In order to inactivate complement components, heat treatment was carried out at 56° C. for 30 minutes. The deactivated components were used for culture.

### (3) Culture Method of Mouse-Derived Ehrlich Ascites Tumor Cell (EATC)

Mouse-derived Ehrlich ascites tumor cells (EATC) were pre-cultured in DMEM medium including 10% FBS for 3 to 4 days in an incubator adjusted to 37° C. and 5% CO₂. Then, the cells were adjusted so that the number of cells was 1.0×10⁶ cells/mL in DMEM medium including 10% FBS, and the sample was added thereto, followed by culturing for 24 hours, and then, the resulting product was subjected to the experiment. To the control group, only ultrapure water was added.

### (4) Culture Method of Mouse Fibroblast (3T3—L1)

3T3—L1 cells as mouse fibroblast were pre-cultured for several days in a medium including 10% FBS, penicillin (50 units/mL, Meiji Seika Kaisha, Ltd.), and streptomycin (50 units/mL, Meiji Seika Kaisha, Ltd.) in an incubator adjusted to 37° C. and 5% CO₂. After cells were exfoliated by treatment with a trypsin/EDTA solution, the number of cells was adjusted to 1.0×10⁵ cells/mL.

Thereafter, as a main culture, the sample was added at the same time as replacement of culture, followed by culturing for 24 hours. Then, measurement was carried out. To the control group, only ultrapure water was added.

### (5) Culture Method of Rat Hepatocytes

Hepatocytes were isolated from the livers of Wistar male rats (body weight: 300 to 350 g) by collagenase perfusion. The cell suspensions collected were diluted 11 fold with 0.4% Trypan blue solution and cell viability was measured. Cells having the cell viability of 90% or more were used for the experiment.

The cells were adjusted in Williams E medium including 10% FBS so that the number of cells was 1.5×10⁵ cells/mL, seeded in 2 mL each in 3.5-cm diameter culture dishes each pre-cultured in an incubator of 37° C. and 5% CO₂ for 24 hours. Thereafter, as a main culture, RNA was added to the medium at the same time as replacement of culture, followed by culturing for 24 hours.

### Test Example 1

It was verified that torula yeast-derived RNA did not affect a normal cell based on the number of living cells.

### (1) Measurement of Number of Living Cells (Neutral Red Method)

Using the neutral red method using characteristics that neutral red that is a red dye, is taken in and accumulated in the endoplasmic reticulum of living cells, the number of living cells was measured as % of Control.

### (2) Reagent

Neutral red (solid reagent), formaldehyde, CaCl₂, CH₃COOH, and EtOH (all of which were manufactured by FUJIFILM Wako Pure Chemical Industries, Ltd.).

(3) Experiment Method (3T3—L1 cell)

In a 3.5-cm diameter dish, 3T3—L1 cells were seeded at 1.0×10⁵ cells/mL, cultured overnight in DMEM medium containing 10% FBS, and the cells were allowed to attach to the culture dish. Then, samples (400 and 800 μg/mL of RNA) were added at the same time as replacement of media, followed by being cultured for 24 hours. After completion of the main culture, the medium was removed, and 1 mL of 0.005% neutral red solution was added, followed by incubation for 2 hours. After 0.005% neutral red solution was removed, the cells were washed with 1% formaldehyde/1% CaCl₂ (2 mL). Successively, 1 mL of 1% CH₃COOH/50% EtOH was added thereto. The resulting product was allowed to stand at room temperature for 30 minutes, and the dye was extracted.

The amount of neutral red absorbed by lysosome was determined by measuring the absorbance of the decolorized extract solution at 540 nm using an absorptiometer (DU 530 by Beckman Coulter).

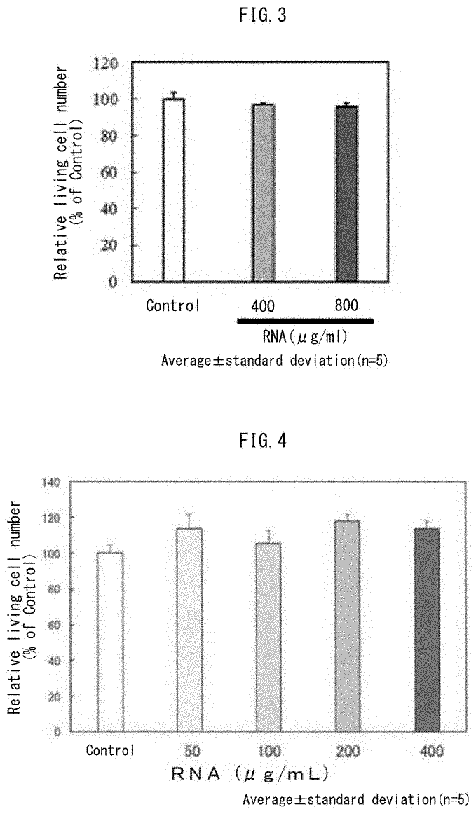

FIG. 3 shows a graph of the effect of RNA on the number of living cells of mouse fibroblasts (3T3-L1, normal cells). From FIG. 3, RNA did not affect the number of living cells at any concentrations.

### (4) Experiment Method (Rat Hepatocytes)

After main culture was completed, the medium was removed, 1 mL of 0.005% neutral red dye solution was added, and the culture was carried out in an incubator at 37° C. and 5% CO₂ for 2 hours. Thereafter, the neutral red dye solution was removed, the cells were washed once with 1% formaldehyde/1% CaCl₂ (2 mL), 1 mL of 1% CH₃COOH/50% EtOH was added thereto. The resulting product was allowed to stand at room temperature for 30 minutes, the supernatant was diluted 3-fold with 1% CH₃COOH/50% EtOH, and the absorbance at 540 nm was measured spectrophotometrically.

FIG. 4 is a graph showing the effect of RNA on the number of living cells of hepatocytes (normal cells). From FIG. 4, it can be considered that the addition of RNA to hepatocytes (normal cells) has no effect on the “Relative living cell number (% of Control)”.

### Test Example 2

An effect of torula yeast-derived RNA on cell viability and cell proliferation potency of EATC was verified.

### (1) Measurement of Number of Cells and Cell Viability (Trypan Blue Method)

Trypan blue cannot permeate the cytoplasmic membrane of living cells and can stain only dead cells. Using this property, Trypan blue was added to a cell suspension, and the number of dead cells and the number of living cells were counted using a Thoma Hemocytometer to calculate the cell viability and cell number.

### (2) Reagent

Trypan blue (FUJIFILM Wako Pure Chemical Industries, Ltd.)

(3) Experiment method

After adding the samples (50, 100, 200, and 400 μg/mL of RNA) and culturing for 24 hours, cells were collected in Falcon tubes cooled in ice. Furthermore, 1 mL of PBS was added to the culture dish, followed by washing, and cells were completely collected. The collected cell suspension in the amount of 50 μL and the equal amount of 0.4% Trypan blue solution were mixed in a 96 well plate and well suspended on a cell chip, followed by cell counting using a Thomas hemocytometer.

Cell viability was calculated as follows.

Cell viability (%)={number of living cells/(number of living cells+number of dead cells)}×100

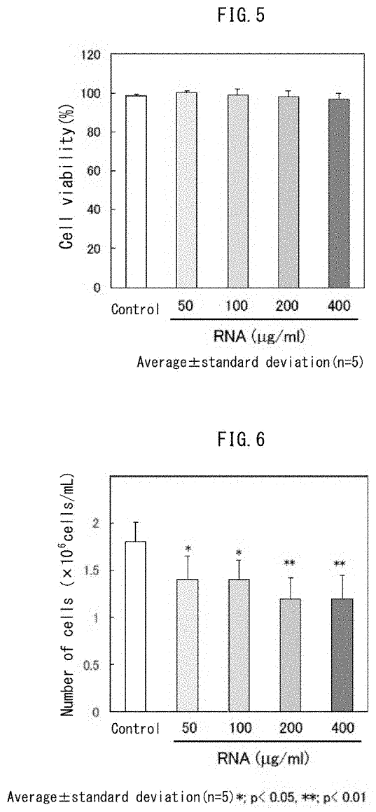

FIG. 5 is a graph showing the effect of RNA on the cell viability of EATC of the present invention. FIG. 6 is a graph showing the effect of RNA on the cell proliferation potency of EATC of the present invention.

From FIG. 5, RNA did not have an effect on the cell viability of EATC (cancer cells). From FIG. 6, however, RNA significantly reduced the number of cells of EATC (cancer cells), demonstrating that RNA had a proliferation suppression effect on cancer cells.

### Test Example 3

### (1) Measurement of DNA Synthesis Ability (BrdU Method)

The BrdU method was carried out to obtain detailed data on how RNA was involved in cell cycle progression. In cell division, DNA as the body of gene in the cell nucleus is replicated. DNA is replicated using compounds called the four bases. One of the bases is thymidine. BrdU (5-Bromo-2′-deoxyuridine) is a thymidine analogue and is taken up by cells in the S phase carrying out DNA synthesis. DNA that has taken up BrdU in the S phase is detected by anti-BrdU antibodies. The percentage of cells synthesizing DNA during BrdU treatment can be demonstrated microscopically.

### (2) Reagent

### BrdU (FUJIFILM Wako Pure Chemical Industries, Ltd.)

0.1% Triton-X[Phosphate-buffered saline solution of polyoxyethylene(10)octylphenyl ether (Fujifilm Wako Pure Chemical Industries, Ltd.)]
Anti-BrdU antibody (primary antibody, Dako Ltd.)
Goat anti-mouse IgG biotin antibody (secondary antibody, Dako Ltd.)

### Streptavidin HRP (Dako Ltd.)

PBS (-) (phosphate buffered saline)
DAB (diaminobenzidine) solution
0.2 M PB (phosphate buffer solution)

### (3) Experiment Method

Into a 3.5-cm diameter culture dish, EATC was seeded at 1.0×10⁶ cells/mL, samples (200 and 400 μg/mL of RNA) were added, followed by culturing for 24 hours. After culturing, 10 μL of 20 mM BrdU solution was added, culturing was carried out for 24 hours. Cells were collected into 15-mL Falcon tube and centrifuged (1000 rpm, 5 minutes, 4° C.). The supernatant was removed, and then, resuspended in 2 mL of PBS (-). The cell suspension was dispensed into microtubes in 1 mL each and centrifuged (10000 rpm, 1 second, 4° C.). PBS (-) was removed, then suspended in PBS (-), and centrifuged again (10000 rpm, 1 second, 4° C.). The supernatant was removed, and 1 mL of EtOh was added, mildly suspended and allowed to stand still at room temperature for 30 minutes and fixed. Cells were centrifuged (10000 rpm, 1 second, 4° C.) and supernatant was removed with 200 mL left, and well suspended. The suspension was dropped onto a glass slide and air-dried.

Thereafter, 100 μL each of 2N HCl was added dropwise, and the resulting mixture was allowed to stand at room temperature for 30 minutes to depolymerize the DNA strands. The depolymerized sample was washed (×twice) with 0.01 M PBS (-), 0.1 M Tris HCl was added dropwise, and allowed to stand at room temperature for 5 minutes. After washing (×twice) with 0.01 M PBS (-), washing with 0.1% Triton-X (×twice) and diluting with 0.01 M PBS (-), a primary antibody (anti-BrdU antibody) was dropped onto a slide glass, and the slide was placed at 4° C. in a wet staining box filled with water while preventing drying.

A secondary antibody (goat anti-mouse IgG biotin antibody) which had been washed with 0.01M PBS(-), and diluted with 0.01M PBS(-) was added dropwise, and allowed to stand still in wet staining box for one hour. Thereafter, streptavidin HRP, which had been washed with 0.01 M PBS(-), and then diluted with 0.01 M PBS(-), was added dropwise, and allowed to stand still for a further one hour. The resulting product was washed with 0.01 M PBS(-), and then immersed in a DAB solution for 5 minutes to develop color. After developing color, it was washed with 0.01 M PBS (-), and a glass slide was covered with a cover glass using water-soluble encapsulant to prepare a specimen. The specimen was examined under a microscope, and cells stained black were counted as BrdU-positive cells (DNA-synthesizing cells) and not-stained cells were counted as BrdU-non-positive cells (DNA-non-synthesizing cells), and the percentage of BrdU-positive cells in the total number of cells was calculated as cells having DNA synthesis ability.

DNA synthesis ability (%)={number of DNA synthesized cells/(number of DNA synthesized cells+number of DNA non-synthesized cells)}×100

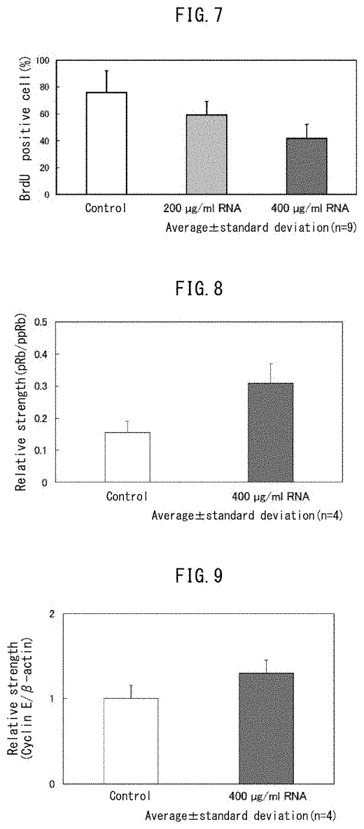

FIG. 7 is a graph showing an effect on the DNA synthesis ability. As compared with the control group, the percentage of BrdU-positive cells was significantly decreased depending on the concentration of RNA, suggesting that the cell cycle of EATC was suppressed in the G1/S phase.

### Test Example 4

### (1) Detection of Cell Cycle Regulatory Protein (Western Blotting)

Western blotting is a method in which a cell lysate prepared using a cell lysis buffer is separated and treated for each molecular weight of protein by using electrophoresis, and transferred to a membrane, and the expression level of a target protein is examined by using an antigen-antibody reaction. The amount of protein in the cell lysate was determined by BCA assay (Thermo Scientific), and the amount of samples loaded into the well was determined.

In this test, 30 μg of protein was separated using SDS-polyacrylamide gel electrophoresis (SDS-PAGE). Fluorescence emission and photographing were carried out using an emission fluorescence imaging system AE-9300 Ez-Capture MG (ATTO CORPORATION). The amount of protein was measured using image analysis software CS Analyzer ver 3.0 (ATTO CORPORATION). As the specimen, 400 μg/mL of RNA was added.

Cell cycle-associated proteins (pRb, cyclin E, p21, and p53) involved in the progression from the G1 phase to the S phase were measured by Western blotting.

### (2) Effect of RNA on Expression of Highly Phosphorylated Rb Protein

When the cell cycle shifts from the G1 phase to the S phase, the Rb protein is phosphorylated to become an inactive form and dissociated from the transcription factor E2F. The activated E2F promotes transcription of the gene required for DNA replication. Whether RNA suppressed phosphorylation of Rb protein and inhibited progression from the G1 phase to the S phase was examined by Western blotting. FIG. 8 is a graph showing an effect of RNA on the expression of highly phosphorylated Rb protein (ppRb). Addition of 400 μg/ml of RNA decreased the amount of high-phosphorylated Rb protein and increased the proportion of low-phosphorylated Rb protein compared with controls, demonstrating that phosphorylation of Rb protein was suppressed.

### (3) Effects of RNA on Cyclin E Protein Expression

Progression of the whole cell cycle is involved in a complex of two proteins, cyclin and cyclin-dependent kinase (Cdk). There are a plurality of types of cyclin/Cdk complexes, promoting the cell cycle by phosphorylating specific substrates at different phases of the cell cycle. After each period, however, they are decomposed by the ubiquitin-proteasome system. Passage through the G1 phase and shift to the S phase are controlled by the cyclin D/Cdk4,6 and cyclin E/Cdk2 complex, which phosphorylate Rb proteins. Since the results showed that phosphorylation of Rb protein was suppressed in the previous section, the effect of RNA on the expression level of cyclin E protein was examined. FIG. 9 is a graph showing an effect of RNA on the expression of cyclin E protein. It was demonstrated that addition of RNA increased the expression level of cyclin E protein as compared with the control group.

### (4) Effect of RNA on p21 Protein Expression

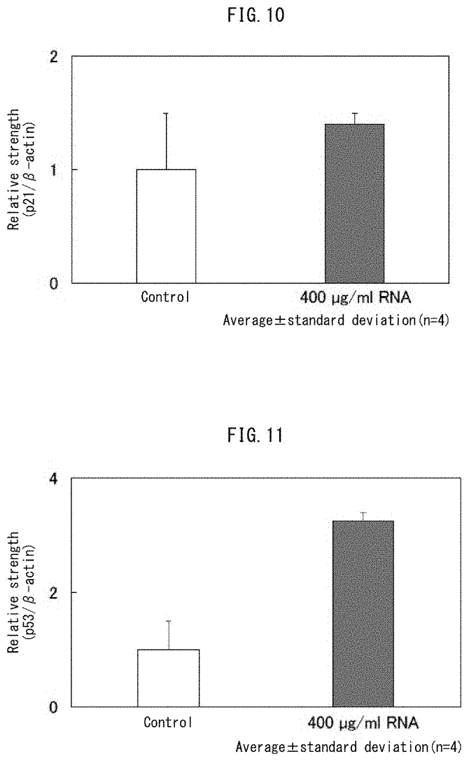

The activity of Cdk or a cyclin/Cdk complex is suppressed by Cdk inhibitory proteins. The cyclin E/Cdk2 complex is controlled by p21 and p27 categorized in the Cip/Kip family. Since the result that addition of RNA increased the expression level of the cyclin E protein was obtained in the previous section, the effect of RNA on the expression level of p21 protein was examined. FIG. 10 is a graph showing the effect of RNA on the expression of the p21 protein. It was demonstrated that addition of RNA increased the expression level of p21 protein as compared with the control group.

### (5) Effect of RNA on p53 Protein Expression

p53 is a representative cancer suppressor gene for which genetic mutation has been detected in many human cancer cells. p53 acts on DNA damage of a cell to increase the expression level of p21, thereby stopping the cell cycle and promoting DNA repair so as to suppress DNA mutations and eliminating not repaired cells by apoptosis. Thereby, cells with genetic mutations are prevented from remaining. Since the result that addition of RNA increased the expression level of p21 protein was obtained in the previous section, the effect of RNA on the expression level of p53 protein was examined. FIG. 11 is a graph showing the effect of RNA on the expression of the p53 protein. It was demonstrated that the addition of RNA increased the expression level of p53 protein as compared with the control group.

### Test Example 5

The above-mentioned test suggested that RNA suppress the cell proliferation by controlling the progression from the G1 phase to the S phase of a cancer cell. In order to demonstrate whether this action is obtained in a level of a living body, a tumor-bearing mouse model was prepared by intraperitoneally administering mouse-derived Ehrlich ascites tumor cell (EATC), and an effect of RNA on the cancer cell when RNA was orally administered was studied.

### (1) Breeding of Animals

ICR male mice (Japan SLC) weighing 28 to 30 g (6 weeks old) were used as test animals. During a period from the time of arrival until the end of the experiment, three mice were housed in each cage and bred with free access to a solid food (Lab MR Stock) and tap water. The breeding room was illuminated with a fluorescent lamp from 8 a.m. to 8 p.m., and the room temperature was maintained at 23±1° C.

After 5 days of pre-breeding, the animals were weighed and divided into two groups: (1) a group to which control+EATC (control) were administered, and (2) a group to which RNA+EATC (RNA) were administered. Each group includes six animals.

Samples (RNA: 0.05 mg/g of body weight) dissolved in distilled water were orally administered at a dose of 200 μL every 2 days from the first day of breeding. To the control group, 200 μL of distilled water was orally administered.

On Day 10 after start of breeding, EATC (0.5×10⁵ cells/500 μL) was intraperitoneally administered to mice to prepare tumor-bearing mice. After EATC was administered, distilled water or samples were orally administered every day and bred for 15 days. During the breeding period, feed (solid feed) and water were fed ad libitum. Breeding and testing of animals were conducted in accordance with the Osaka City University Animal Experiment Management Regulations.

(2) Measurement of ascites amount

With the progression of cancer, the abdomen begins to swell. In this experiment, ascites in the peritoneal cavity was aspirated with a syringe at the time of dissection and the amount was measured.

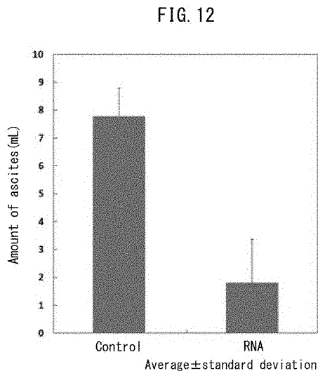

FIG. 12 is a graph showing an effect of RNA on an amount of mouse ascites. Intake of RNA significantly reduced the amount of mouse ascitic fluid as compared with controls, demonstrating that cancer progression was suppressed by remarkably reducing cancer cells.

1. A cancer cell proliferation inhibitor which is a formulation inhibiting progression of a cell cycle of a cancer cell, comprising a yeast-derived RNA as an active component.

2. The cancer cell proliferation inhibitor according to claim 1, wherein the yeast-derived RNA is an RNA extracted from torula yeast.

3. The cancer cell proliferation inhibitor according to claim 1, wherein the yeast-derived RNA is an RNA inhibiting progression from a G1 phase to an S phase in the cell cycle of the cancer cell.

4. A health food for inhibiting progression of a cell cycle of a cancer cell, comprising a yeast-derived RNA as an active component.

5. The health food according to claim 4, wherein the yeast-derived RNA contains an RNA extracted from torula yeast.

6. The health food according to claim 4, wherein the yeast-derived RNA contains an RNA inhibiting progression from a G1 phase to an S phase in the cell cycle of the cancer cell.
